(19) United States
(12) Patent Application Publication
(10) Pub. No.: US 2018/0280199 A1
(43) Pub. Date: Oct. 4, 2018

(54) NOISE REDUCTIONS DEVICE

(71) Applicant: MICRONOISE LTD, Tel-Aviv (IL)

(72) Inventor: Tsahi MERKUR, Tel-Aviv (IL)

(21) Appl. No.: 15/770,219

(22) PCT Filed: Oct. 16, 2016
(86) PCT No.: PCT/IL2016/051124

§ 371 Date: Apr. 23, 2018

Related U.S. Application Data
(60) Provisional application No. 62/244,210, filed on Oct. 21, 2015.

(51) Int. Cl. A61F 11/08; G10K 11/172

(52) U.S. Cl. CPC A61F 11/08; A61F 2011/085; G10K 11/172

(57) ABSTRACT

A noise reductions device is provided the device includes an outer member having an inlet opening and an outlet opening; a hollow spherical member mounted in the opening and configured for mixing sound waves; a cannula having an inlet tip in communication with the hollow spherical member, and an outlet tip configured for transmitting sound waves therethrough; wherein the cannula includes at least one winding between the inlet tip and the outlet tip.

### FIELD OF INVENTION

The presently disclosed subject matter relates to noise reductions device for reducing noise, in general, and in particular to a noise reductions device for insertion in a human ear.

### BACKGROUND

The need for adequate hearing protection in high noise environments has long been recognized among those concerned with health and safety issues, and much effort has gone into providing such protection.

There are mainly four types of earplugs for hearing protection: Foam earplugs, mainly made of memory foam, which are compressed and put into the ear canal, where they expand to plug it. Silicone earplugs, which are rolled into a ball and carefully molded to fit over the external portion of the ear canal. Flanged earplugs, including most types of musicians' or ‘Hi-Fi’ earplugs. Custom molded earplugs, made from a mold of the wearer's ear and designed to precisely fit all ear canal shapes. Custom molded is further divided into Laboratory made and “formed in place”.

Earplugs are especially useful to people exposed to excessively noisy devices or environments (80 dB or more).

The human ear was not created to withstand levels of sound and noise, which are only crumbs of sound produced by devices invented in this century.

Much has been said, much invented and much investigated in the academic field in order to achieve protection against this nuisance—the general noise syndrome.

It should be stressed that all instrumentation invented for this purpose was defined for various frequency ranges, which our ear can and is desirous of hearing (music and speech). There is no definition for the range of noise frequencies, apart from planned recorded noise which is not natural in the real noise environment. Noise as such does not suit our ears, which were not designed to receive it.

Frequency is cycles per second times 2, the human ear was created for receiving external information communicated between people and between people and the environment. Sound is a wave of physiological structure in nature, produced by people, animals, the forces of nature or instrumentation. Sound frequencies humans can hear range from 1 to 16 Hz and from 16 to 16,000 Hz. This sound carries from any physiological or mechanical structure in its natural environment, as follows:

From 1 to 16 Hz we have what is known as sensed frequencies, received by all crawling animals which are in contact with rigid surfaces, such as the ground, trees, etc., as well as all living creatures, including humans, which receive their frequencies, in their bodily structure, their bones, and mainly in humans by means of the eardrum. These frequencies are defined as under-infrasound.

From 16 to 125 Hz we have the alarm and warning frequencies, which activate biological alarm systems and liquid secretion systems for strengthening the resistance to stress and fear situations and aggressive phenomena and distortion of the coordinative system in directing equilibrium and chemical balance, secreted in the inner ear and required for function of nervous system and the brain. For example, the semicircular canals transmit commands to the brain and for the general dominant balance of our body and soul.

From 125 to 3000 Hz we have so-called supersound, which is recorded in the scheme of counter-octave for the piano, plus three octaves, 1,2,3, these being considered as central auditory frequencies for listening to fine music, the human voice, and voices of animals.

From 3000 to 16,000 Hz we have the range of “power sound” covering human speech and music. These are high frequencies produced by high-octave musical instruments, by various animals and howling creatures, and also produced by metals and tools being rubbed together.

Measuring instruments developed in order to test instrumentation existing today do not answer these needs, compared to the human ear, in order to supply the diagnosis of protective systems for the hearing.

It should be noted that existing devices, such as noise-blocking ear caps, noise-blocking earplugs, foam-stoppers and electronic phase-cancellation instruments, intended to stop up the ear much like a bottle or a dump box, do not help.

In a world convention held in 1972 on noise safety, in which the aforesaid devices were discussed, the U.S.A., for example, was reported to have 20 million people with noise damage, i.e., about 10% of the entire population. A similar percentage was observed all over the world. Today the statistics indicate that this proportion has almost doubled, due to growing sophistication and intensity of noise, military, industrial, environmental, motor-car produced. It must be stressed and understood that in the foregoing we have explained what sound is. The intensity is the production of pressure, or air, or amplification which lands on a persons ear.

In the hearing examination chart and in these accessories so far built, we encounter attenuation curves connected to frequencies of 125 Hz and below. Nor has any solution been found, so that even more severe damage is inflicted, amounting sometimes to total deafness. It is noted that, in view of the hearing chart, the ratios between frequency intervals are one of the order of thousands, as miserable and poor chronological ratio existing within the hearing organ—no less than 16,000 individual capillaries.

At the Strasbourg Convention on Hearing Safety in 1996, a warning was issued of a possible disaster of the increased deafness among youngsters, children, even babies, reaching the proportions of a syndrome, all due to noisy music, Walkman devices and civil conditions. Other effects, potentially disastrous, which force us to provide total protection for the auditory system are pressures produced by the terrestrial globe under its atmospheric envelope. The eardrum, mechanical and liquid hearing system operates on account of differences in air pressure. The force producing sound is air pressure. Noise is not well received by the human ear, which must therefore be protected from it.

In the Strasbourg Report it was stated that in 1996, in Europe alone, about 20-50 billion dollars were spent on treating and combating this situation. At the same time, a European Directive was accepted, on French initiative, based on a statistical report forecasting deafness and hearing impairment in the next generation.

But in contrast, the academic world of ear specialists agrees with the biological structure of the ear, giving rise to paradox: the external ear of humans or animals, which receives the external sound messages, is not built to be stopped up. It is a living ear, having secretions and needing ventilation, which may be true up to a point, but the air pressure acted in both directions. The air in the external ear canal tried to get out, but the air driven by the external noise and sound found its way in quite readily.

### SUMMARY OF INVENTION

There is provided in accordance with an aspect of the presently disclosed subject matter a noise reductions device. The device includes an outer member having an inlet opening and an outlet opening; a hollow spherical member mounted in the opening and configured for mixing sound waves; a cannula having an inlet tip in communication with the hollow spherical member, and an outlet tip configured for transmitting sound waves therethrough; wherein the cannula includes at least one winding between the inlet tip and the outlet tip.

The outer member can be a cylindrical member. The cylindrical member can include a conical portion defined at the end thereof such that the outlet opening can be defined at the end of the conical portion. The outlet tip can be in fluid communication with the outlet opening.

The noise reductions device can further include a circular membrane mounted on the outlet opening and being configured for sealing engagement between the outlet opening and an external ear.

The circular membrane can be configured with an opening for extending therethrough the outlet opening.

The cannula can include a plurality of winding between the inlet tip and the outlet. A radius of the winding can be 4-6 millimeter.

The outer member can be configured for mounting inside a human ear. A diameter of the hollow spherical member can be 10-14 millimeter.

A length of the conical portion can be at least one third of the length of the cylindrical member.

### BRIEF DESCRIPTION OF THE DRAWINGS

In order to understand the disclosure and to see how it may be carried out in practice, embodiments will now be described, by way of non-limiting examples only, with reference to the accompanying drawings, in which:

FIG. 1 is a perspective view of a noise reduction device in accordance with an example of the presently disclosed subject matter;

FIG. 2A is a perspective view of a cylindrical member of the noise reduction device of FIG. 1A;

FIG. 2B is a perspective view of a coiled cannula member of the noise reduction device of FIG. 1A in accordance with an example of the presently disclosed subject matter;

FIG. 3A is a perspective view of a circular membrane of the noise reduction device of FIG. 1A;

FIG. 3B is a side view of a coiled cannula member of the noise reduction device of FIG. 1A in accordance with an example of the presently disclosed subject matter; and

FIG. 3C is a side sectional view of an opening inlet of the coiled cannula member of the noise reduction device of FIG. 1A in accordance with an example of the presently disclosed subject matter.

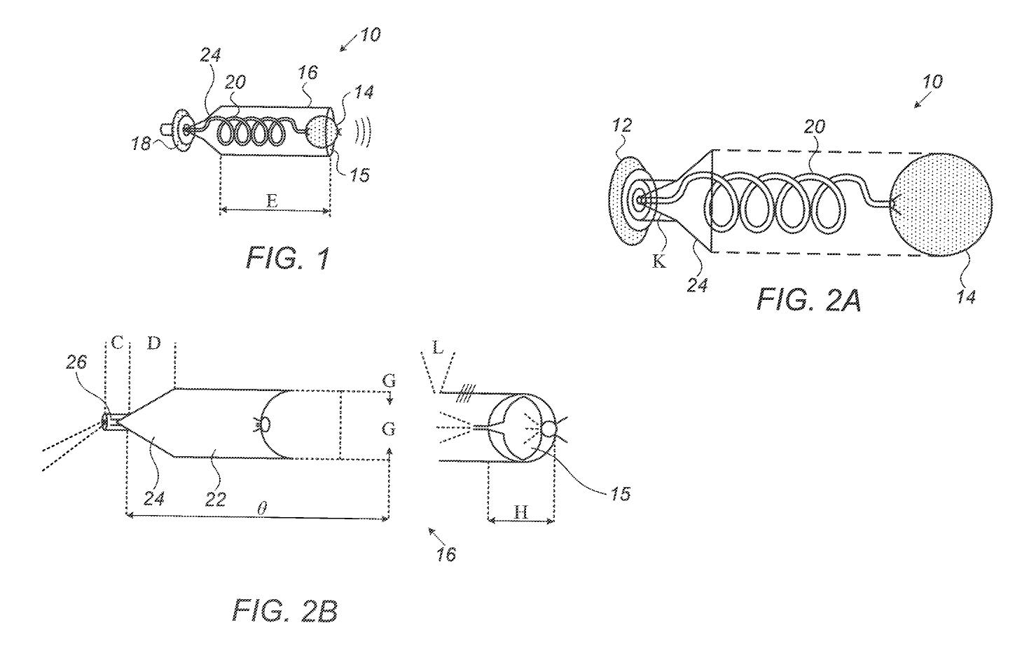

### DETAILED DESCRIPTION OF EMBODIMENTS

The aforesaid system, subject of the invention, in order to (a) enable protection against noise and simultaneously permit one to hear music and speech, for the sake of communicating, functioning and understanding under such conditions; (b) to protect the biological system in all of the ear; (c) to balance the right and left ear in cases of blocking, colds, illness, etc.; (d) to ventilate and direct internal systems (external, middle and inner) in order to prevent inflammation, secretions and disfunctions or to protect an auditory system already damaged, by past exposure to dangerous noise, in the mechanical or nervous systems, in order to prevent further deterioration.

The present invention thus provides a noise reduction device which according to an example can be configured for mounting in the ear. The noise reduction device is configured for high sound attenuation for example by reducing the amplitude of a sound wave to a predetermined level. The device can be configured to reduce noise to a predetermined level, such that other noises below the predetermined level are not affected.

As shown in FIG. 1, the device 10 according to an example includes a cylindrical member 16 having an inlet opening 15 opened to the ambient and an outlet opening 26 open to the ear of the user. The device 10 further includes a hollow spherical bubble 14 which acts as a mixer and which is mounted at the inlet opening 15 of the cylindrical member 16. The cylindrical member 16 can be configured to be inserted inside an ear canal.

The device 10 further includes a coiled cannula 20 disposed inside the cylindrical member 16, and having a first tip 3a, a second tip 23b, and a coiled portion 25 therebetween, The cannula 20 defines a sound wave path therein extending between the first tip 23a and the second tip 23b. The first tip 23a of the coiled cannula 20 is coupled to the spherical bubble 14 is in open thereto, while the second tip 23b is opened to outlet opening 26 of the cylindrical member 16. This way, air inlet from the inlet opening into the cylindrical member is carried out only through the coiled cannula 20.

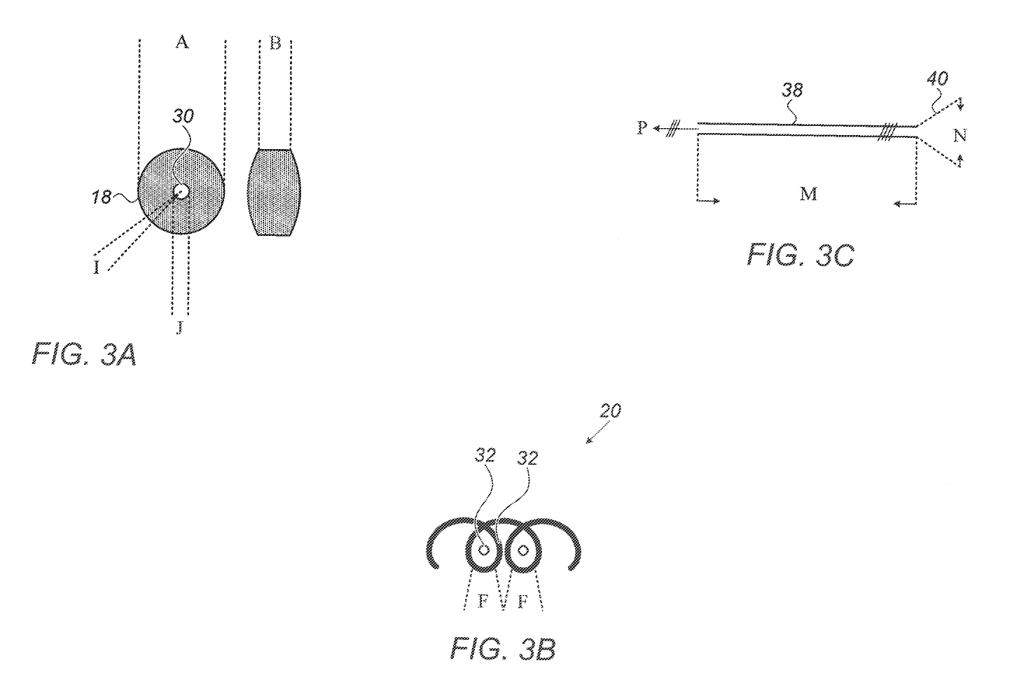

As shown in FIG. 3C, the inlet tip can include a conic opening 40 in communication with an elongated extension 38, such that the conic opening is configured to collect sounds waves from the hollow spherical bubble 14.

The device 10 further includes a conical portion 24 defined at the end of the cylindrical member 16 such that the outlet opening 26 is defined on the conical portion 24 and can be inserted inside the ear. The device 10 further includes a circular membrane 18 mounted about the outlet opening 26 and configured for sealing engagement with the ear skin.

The circular membrane 18 serves as blocking seat in the opening entrance to the external ear, and an inner air-entry passage. The circular membrane 18 is mounted on the conical portion 24 and is provided with an opening sized to allow extending tip of the outlet opening 26 therethrough. The spherical bubble 14, which is provided with an entrance passage for external air is coupled to the coiled cannula 20 and allows air passage into the ear after passing through a number of windings of the cannula 20.

As shown in FIG. 3B, the winding can be configured with a specific radius 32, as explained hereinafter.

The device 10 functions as an air-pressure reservoir for the entire system, and as a counter-wall to the eardrum, which on its other side, towards the middle ear with all its components (malleus, incus, steps oval window, round window). That is to say, the device can be configured to regulate the pressure inside the ear protecting thereby form sudden pressure gradient and shock waves. The device 10 is intended to function as an acoustic resonance box, to amplify sounds of the music and speech center the result is in accordance with the desired wavelength at the frequencies i.e. from 250 to 2500 Hz.

Mounted on the cylindrical member 16 and concentrates resonance in a vestibule in which the tip of the cannula 20 is positioned, in front of the outlet opening 26. The cannula 20 receives sound waves from the spherical bubble 14 which acts as a mixer, vibrates itself, and lets flow its power according to a spiral relationship: dBx radius, i.e., 10 dB per spiral. The outlet opening 26 whence it discharges towards the ear and resonates at frequencies from 2000 to 5000 Hz, thus amplifying the richness of hearing in the speech and music range. The membrane 18 is configured to block air which does not arrive through the device 10 and serves as radio phonic shock-absorber between the auditory system and the bone conduction and as a brake on various air pressures.

The device structure in general, due to its volume, length and various shapes, firms an acoustic vessel for the development, coordination, sorting and separation of sound having penetrated its composition of the various materials used, in relation to the range of frequencies as exactly needed and desirable. The device structure may differ in dimensions, as required for individual needs.

The device is bi-directional and releases vented pressures towards the entrance, in correspondence with the space of the middle ear, which will release its various pressures, liquids, airs, which are collected in it from the inner ear through the round window and to the valve of surplus into the gullet.

It is appreciated that the device can be made of various injection-molded plastics; without any electronic. Alternatively, the device can be also attached to electronic devices; sealants-sponge-foam rubber of suitable grade.

Dimensions of the device can be as follows:
A. 13 MM
B. 7 MM
C. 7 MM
D. 12 MM
E. 31 MM
F. 5 MM
G. 21 MM
H. 12 MM
I. 1.5 MM
J. 8 MM
K. 8 MM
L. 1 MM
M. 35 MM/0.30 MM
N. 0.65 MM
O. 35 MM
P. 0.30 MM

Wherein the letters A-P designate various dimensions of the device as shown in the figures.

The sound wave path in the cannula can be a hollow path or having a sound wave conducting content therein.

The coiled portion can include one or more windings, such that the sound wave travels inside the cannula before reaching the inner volume of the cylindrical member. According to an example, the number of windings determines the attenuation rate of the device. It is appreciated that according to an example various portions of the device can be made of materials configured for noise reduction, such that theses portions are utilized for sound attenuation in conjunction with the coiled portion. It is further appreciated that the properties of these material can be selected so as to determine the desired level of attenuation.

Although the cannula is described here as a coiled cannula having a plurality of windings, according to other examples the cannula can include a plurality of curves, which are not winded about a single axis, but rather curved in different directions.

According to an example, the diameter of the inner sound wave path of the cannula is significantly smaller than the diameter of the cylindrical member, such that the propagation of the sound wave inside the cannula is different than the propagation thereof inside the cylindrical member. It is appreciated that according to other examples the cannula can include a polygonal cross section, as opposed to a cylindrical cross section, such as tetragon, pentagon hexagon and so on.

The device of the invention can be configured to provide a total blocking with minimum contact with the external ear, anterior to the canal entry. The device can further provide an equal ventilation of the external ear and the canal, up to the auditory membrane, equalization of reception of bone frequencies (radiophone) and their balancing in bone-hearing. The device of the invention can he configured for arresting, neutralizing and cancelling any noise of any frequency.

The device can be configured to replace an external ear. According to hearing theory, hearing keeps naturally deteriorating down to minus 50 to minus 70 dB. If the middle ear has been completely neutralized, as made possible by the present invention, then according to the laws of audiometric academy, a person without a middle ear could hear high frequencies from minus 50 to minus 70 dB, transmitted through the auditory and skull bones. According to an example the outer member can be configured to impulse noise reduction of about 50 db.

According to an example, the cannula can include more than one cannulas, or can include more than one opening, that is to say, the first tip or the second tip of the cannula can be split to two or more tips each having an opening. This way inlet air into the cannula or outlet air out of the cannula can be carried out through more than one openings. According to a further example the cannula can include further opening along the length thereof i.e. between the first and second tips thereof.

The outlet opening 26 is configured to be deposed inside the outer ear, such that air from the cylindrical member in the ear, and vice versa, is be carried out though the outlet opening.

The circular membrane 18 can be made of rubber or any other self-conforming material. The circular membrane 18 is configured to seal the periphery of the cylinder, such that air enters the ear only through the cannula and the inner volume of the cylinder and substantially no air can enter the ear from the periphery of the cylindrical member.

It is appreciated that according to an example the device described hereinabove does not define the level of attenuation (e.g. 20 db) but rather blocks ALL noises at a given level. In other words the device can be configured to limit noise to a predetermined level (e.g. 78 db), thus preventing any noise loader than the predetermined level from reaching the inner ear.

It is appreciated that the sound attenuation provided by the device allows carrying out regular conversation and even whispering while using the device. In addition the device allows significant air circulation between the inner ear and the device itself in a way that support long term use.

Although according to the above example the noise reduction device is configured for mounting in the ear, according to other examples the noise reduction device can be configured to be mounted around the ear or can be configured to be mounted on audio devices, such as a telephone speaker etc. It should be noted that the device can be mounted on a speaker of a telephone device or alternatively on the microphone thereof. Accordingly, the cannula can mounted inside a member which is not cylindrical but rather has any other shape. For example the member in which the cannula is mounted can be configured to be installed inside an audio device, such as a microphone, or speaker, etc.

According to an example the noise reduction device of the present invention allows reducing decibel to a predetermined decibel level.

According to an example the device as described hereinabove affects the auditory system by synchronized blocking of the middle ear and controlling the pressure in the inner ear.

The device can be utilized for noise reduction in low-medium noise environment for example in cases where low frequency sound becomes the cause for systemic disease

The device can be further utilized for improving audio communication experience and for precluding exposure to undesirable noises which may cause tension. For example, for people who reside in close proximity to airports, heavy traffic etc.

Those skilled in the art to which the presently disclosed subject matter pertains will readily appreciate that numerous changes, variations, and modifications can be made without departing from the scope of the invention, mutatis mutandis.

1. A noise reductions device comprising:
an outer member having an inlet opening and an outlet opening;
a hollow spherical member mounted in said opening and configured for mixing sound waves;
a cannula having an inlet tip in communication with said hollow spherical member, and an outlet tip configured for transmitting sound waves therethrough;
wherein said cannula includes at least one winding between said inlet tip and said outlet tip.

2. The noise reductions device of claim 1 wherein said outer member is a cylindrical member.

3. The noise reductions device of claim 2 wherein said cylindrical member includes a conical portion defined at the end thereof such that the outlet opening is defined at the end of said conical portion.

4. The noise reductions device of claim 3 wherein said outlet tip is in fluid communication with said outlet opening.

5. The noise reductions device of claim 1 further comprising a circular membrane mounted on said outlet opening and being configured for sealing engagement between said outlet opening and an external ear.

6. The noise reductions device of claim 1 wherein said circular membrane is configured with an opening for extending therethrough said outlet opening.

7. The noise reductions device of claim 1 wherein said cannula includes a plurality of winding between said inlet tip and said outlet tip.

8. The noise reductions device of claim 1 wherein a radius of said winding is 4-6 millimeter.

9. The noise reductions device of claim 1 wherein said outer member is configured for mounting inside a human ear.

10. The noise reductions device of claim 1 wherein diameter of said hollow spherical member is 10-14 millimeter.

11. The noise reductions device of claim 3 wherein a length of said conical portion is at least one third of the length of said cylindrical member.
